(19) United States
(12) Patent Application Publication
(10) Pub. No.: US 2007/0093724 A1
(43) Pub. Date: Apr. 26, 2007

(54) AUTOMATIC APNEA/HYPOPNEA DETECTION DEVICE, DETECTION METHOD, PROGRAM AND RECORDING MEDIUM

(75) Inventor: Hiroshi NAKANO, Irvine, CA (US)

Correspondence Address: KNOBBE MARTENS OLSON & BEAR LLP, 2040 MAIN STREET, FOURTEENTH FLOOR, IRVINE, CA, 92614, US

(73) Assignee: Takeshi TANIGAWA, Tsukuba (JP)

(21) Appl. No.: 11/425,329

(22) Filed: Jun. 20, 2006

(30) Foreign Application Priority Data
Oct. 11, 2005 (JP) 2005-296849

(51) Int. Cl. A61B 5/08

(52) U.S. Cl. 600/538; 600/529; 128/204.23

(57) ABSTRACT

A device includes a respirometer 3 and an automatic apnea/hypopnea analyzer 2. The respirometer 3 includes an airflow-signal recording unit 13 that is connected to a thermistor respiratory flow meter 5 or a nasal-pressure type flow meter 6 detecting the airflow waveform signals, converts to digital data the airflow waveform signals obtained by the thermistor respiratory flow meter 5 or the nasal-pressure type flow meter 6, and stores the converted data as measured respiratory flow values. The automatic apnea/hypopnea analyzer 2 obtains power spectra in the breathing frequency band from the measured respiratory flow values, calculates logarithmic time-series data from the power spectra, smoothes the data, and then detects transitory drops (flow power dips) in the smoothed data, to automatically detect apnea/hypopnea.

### BACKGROUND OF THE INVENTION

1. Field of the Invention

The present invention relates to a reliable automatic apnea/hypopnea detection device, detection method, program and recording medium, in respiratory monitoring using a single channel.

2. Description of the Related Art

The sleep apnea/hypopnea syndrome has become a social issue in recent years. The sleep apnea/hypopnea syndrome is a disease that is suffered by at least several percent of the entire population. The patient with this syndrome is prevented from sufficient sleep due to repeated occurrences of apnea (halting of breathing) or hypopnea (decrease of breathing volume). These symptoms also place significant loads on the cardiovascular system.

The sleep apnea/hypopnea syndrome is an important issue that not only affects the health of individual patients (drowsiness, lower work efficiency, triggering of cardiovascular disorder), but may also cause social problems such as traffic accidents. In Japan, this syndrome has been widely recognized in recent years, and organizations are starting to conduct mass-screening for the sleep apnea/hypopnea syndrome.

Home respiratory monitoring is used as a means for screening the sleep apnea/hypopnea syndrome as part of physical checkup. However, the breathing sensor used in home respiratory monitoring does not by itself ensure sufficient reliability of analysis results, because the signal levels change easily due to the change in breathing route, shifting of sensor position, and so on.

To improve this drawback, multi-channel simultaneously measurement is performed in which multiple sensors are attached to several locations of the patient's body (Japanese Patent Laid-open No. Hei 5-200031).

### SUMMARY OF THE INVENTION

However, the invention disclosed in Japanese Patent Laid-open No. Hei 5-200031 cannot be used in large-scale mass examinations, partly due to the cumbersome procedure associated with the attachment of sensors to several locations of the patient's body, and partly due to the need for visual analysis that requires a lot of manpower.

An object of the present invention is to solve one or more of the aforementioned problems inherent in the conventional method, by realizing a reliable automatic apnea/hypopnea detection device, detection method, program and recording medium in respiratory monitoring using a single channel.

To solve one or more of the aforementioned problems, an embodiment of the present invention provides an automatic apnea/hypopnea detection device comprising a respirometer and an automatic apnea/hypopnea analyzer and automatically detecting apnea/hypopnea based on the airflow waveforms of inhalation and exhalation resulting from breathing of the subject, wherein the respirometer comprises a flow meter that detects the airflow waveform signals, and an airflow-signal recording unit that converts the airflow waveform signals to digital data as measured values; and wherein the automatic apnea/hypopnea analyzer comprises: a means for, by obtaining power spectra from the measured values by Fourier conversion, obtaining time-series data of flow power covering the total of all power spectra belonging to the breathing frequency band among the obtained power spectra as well as time-series data of noise power covering the total of all power spectra belonging to the non-breathing frequency bands among the obtained power spectra, while obtaining logarithmic time-series data of flow power and logarithmic time-series data of noise power from the time-series data of flow power and time-series data of noise power by logarithmic conversion; a means for smoothing the logarithmic time-series data of flow power; a means for detecting a flow power dip, which is a transitory drop in the smoothed logarithmic time-series data of flow power; a means for certifying whether the logarithmic time-series data of flow power before smoothing is valid or invalid under specified conditions; a means for automatically excluding flow power dips that generated in an invalid section of the logarithmic time-series data of flow power; and a means for detecting the number of flow power dips occurring per unit valid period of the logarithmic time-series data of flow power, and thereby automatically detecting apnea/hypopnea.

The means for certifying whether the logarithmic time-series data of flow power before smoothing should ideally have a structure to certify whether the logarithmic time-series data of flow power before smoothing is valid or invalid under the conditions that the flow power is equal to or above a specified level and the ratio of the flow power and noise power is equal to or above a specified value, and register as an invalid data section any section of logarithmic time-series data of flow power not satisfying the conditions from among the flow power dips.

The flow meter should ideally be a thermistor respiratory flow meter or a nasal-pressure type respiratory flow meter.

To solve one or more of the aforementioned problems, an embodiment of the present invention provides a method of automatically detecting apnea/hypopnea that detects breathing of the subject using a respirometer and automatically analyzes the digitally converted measured values using an automatic apnea/hypopnea analyzer to automatically detect apnea/hypopnea, wherein the method for automatically detecting apnea/hypopnea comprises: a step for, by obtaining power spectra from the measured values by Fourier conversion, obtaining time-series data of flow power covering the total of all power spectra belonging to the breathing frequency band among the obtained power spectra as well as time-series data of noise power covering the total of all power spectra belonging to the non-breathing frequency bands among the obtained power spectra, while obtaining logarithmic time-series data of flow power and logarithmic time-series data of noise power from the time-series data of flow power and time-series data of noise power by logarithmic conversion; a step for smoothing the logarithmic time-series data of flow power; a step for detecting a flow power dip, which is a transitory drop in the smoothed logarithmic time-series data of flow power; a step for certifying whether the logarithmic time-series data of flow power before smoothing is valid or invalid under specified conditions, and registering as an invalid data section any section of logarithmic time-series data of flow power not satisfying the conditions, a step for automatically excluding, among the flow power dips, those flow power dips that generated in the invalid data sections; and a step for calculating a respiratory disturbance index as the number of flow power dips occurring per unit time of valid sections excluding the invalid sections.

In the method of automatically detecting apnea/hypopnea, the specified conditions should ideally be that the flow power is equal to or above a specified level and that the ratio of the flow power and noise power is equal to or above a specified value.

To solve one or more of the aforementioned problems, an embodiment of the present invention provides an automatic apnea/hypopnea detection program that is installed in a computer so that it detects breathing of the subject using a respirometer and automatically analyzes the digitally converted measured values in order to automatically detect apnea/hypopnea, wherein the automatic apnea/hypopnea detection program causes the computer that automatically analyzes apnea/hypopnea to function as: a means for, by obtaining power spectra from the measured values by Fourier conversion, obtaining time-series data of flow power covering the total of all power spectra belonging to the breathing frequency band among the obtained power spectra as well as time-series data of noise power covering the total of all power spectra belonging to the non-breathing frequency bands among the obtained power spectra, while obtaining logarithmic time-series data of flow power and logarithmic time-series data of noise power from the time-series data of flow power and time-series data of noise power by logarithmic conversion; a means for smoothing the logarithmic time-series data of flow power; a means for detecting a flow power dip, which is a transitory drop in the smoothed logarithmic time-series data of flow power; a means for certifying whether the logarithmic time-series data of flow power before smoothing is valid or invalid under specified conditions, and registering as an invalid data section any section of logarithmic time-series data of flow power not satisfying the conditions; a means for automatically excluding, among the flow power dips, those flow power dips that generated in the invalid data sections; and a means for calculating a respiratory disturbance index as the number of flow power dips occurring per unit time of valid sections excluding the invalid sections.

In the automatic apnea/hypopnea detection program, the specified conditions should ideally be that the flow power is equal to or above a specified level and that the ratio of the flow power and noise power is equal to or above a specified value.

To solve one or more of the aforementioned problems, an embodiment of the present invention provides a recording medium that can be read by a computer, in which the automatic apnea/hypopnea detection program is stored.

Effects of the Invention

The automatic apnea/hypopnea detection device, detection method, program and recording medium pertaining to the above embodiments of the present invention provide at least one of the following effects:

1) Apnea/hypopnea can be detected automatically via a simple structure by detecting as airflow waveforms the change of airflow (respiratory flow) generated by breathing, obtaining from these airflow waveforms the power spectra in the breathing frequency band as a function of time, obtaining time-series data of their logarithms, and then recognizing a pattern of transitory drops in the logarithmic data (flow power dips).

2) Also, the present invention achieves automatic detection of apnea/hypopnea with high reliability, because the validity of measured respiratory flow values is certified based on power spectrum values, and invalid sections are automatically excluded.

For purposes of summarizing the invention and the advantages achieved over the related art, certain objects and advantages of the invention have been described above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

Further aspects, features and advantages of this invention will become apparent from the detailed description of the preferred embodiments which follow.

### BRIEF DESCRIPTION OF THE DRAWINGS

FIG. 1 is a drawing explaining the overall structure of an automatic apnea/hypopnea detection device pertaining to the present invention used in an example.

FIG. 2 is a drawing explaining a thermistor airflow sensor and an airflow-signal recording unit constituting a respirometer shown in the above example of the present invention.

FIG. 3 is a drawing showing a frontal view of a nasal-pressure type airflow sensor constituting the respirometer shown in the above example of the present invention.

FIG. 4 is a flowchart explaining the structure and operation of the example using the automatic apnea/hypopnea detection device, detection method, program and recording medium pertaining to the present invention.

FIG. 5 shows various data obtained by the automatic apnea/hypopnea detection device, detection method, program and recording medium pertaining to the present invention.

FIG. 6 is another set of various data obtained by the automatic apnea/hypopnea detection device, detection method, program and recording medium pertaining to the present invention.

FIG. 7 shows various data obtained by the automatic apnea/hypopnea detection device, detection method, program and recording medium pertaining to the present invention.

FIG. 8 shows various data obtained by the automatic apnea/hypopnea detection device, detection method, program and recording medium pertaining to the present invention.

FIG. 9 shows various data obtained by the automatic apnea/hypopnea detection device, detection method, program and recording medium pertaining to the present invention.

Description of the symbols: 1: Automatic apnea/hypopnea detection device; 2: Automatic apnea/hypopnea analyzer; 3: Respirometer; 4: Printer; 5: Thermistor respiratory flow meter; 6: Nasal-pressure type respiratory flow meter; 7: Thermistor airflow sensor; 8: Sensor; 9: Nostril outlet; 10: Oral slit; 11: Thermistor element; 12: Lead wire; 13: Airflow-signal recording unit; 14: Input terminal of airflow-signal recording unit; 15: A/D converter; 16: Control device; 17: Memory of airflow-signal recording unit; 18: Output part; 19: Power supply; 20: Nasal-pressure type airflow sensor; 21: Nasal tube; 21′: Hollow pipe; 22: Opening of nasal tube; 23: Pressure transducer; 24: I/O interface; 25: Bus of automatic apnea/hypopnea analyzer; 26: CPU of automatic apnea/hypopnea analyzer; 27: Memory of automatic apnea/hypopnea analyzer.

### DETAILED DESCRIPTION OF THE PREFERRED EMBODIMENT

An embodiment of the automatic apnea/hypopnea detection device, detection method, program and recording medium in which this program is recorded, pertaining to the present invention, is explained below using an example by referring to the drawings. The examples and drawings are not intended to limit the present invention.

The automatic apnea/hypopnea detection device, detection method, program and recording medium pertaining to an embodiment of the present invention are characterized by the automatic detection of apnea/hypopnea achieved by using a respirometer to detect airflow waveforms generated by breathing of the subject, converting them to digital data and using the obtained measured values to calculate the power spectra in the breathing frequency band as a function of time (time-series data of flow power), converting the data to logarithms (logarithmic time-series data of flow power), smoothing the logarithmic time-series data, and then detecting transitory drops (“flow power dips”) in the smoothed data.

The automatic apnea/hypopnea detection device, detection method, program and recording medium are also characterized by the high reliability achieved by certifying the validity of measured respiratory flow values based on flow power and noise power values, and automatically excluding flow power dips in invalid sections.

In the present disclosure where conditions and/or structures are not specified, the skilled artisan in the art can readily provide such conditions and/or structures, in view of the present disclosure, as a matter of routine experimentation.

### EXAMPLE

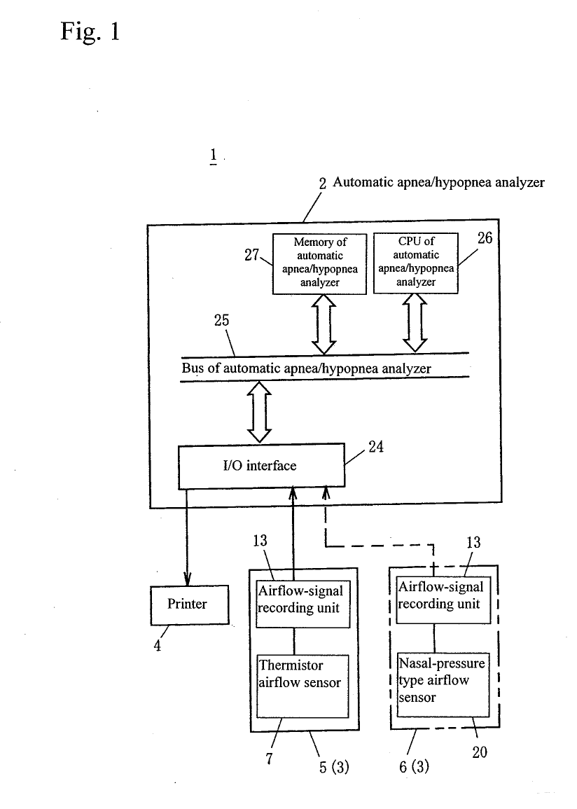

FIG. 1 explains the overall structure of an automatic apnea/hypopnea detection device 1 pertaining to an embodiment of the present invention. The automatic apnea/hypopnea detection device 1 comprises an automatic apnea/hypopnea analyzer 2 and a respirometer 3. The automatic apnea/hypopnea analyzer 2 can be connected to an output device, such as a printer 4, to output, as necessary, the detected data obtained from the automatic apnea/hypopnea detection device 1.

As the respirometer 3, a thermistor respiratory flow meter 5 or a nasal-pressure type respiratory flow meter 6 (indicated inside the imaginary border in FIG. 1) is used.

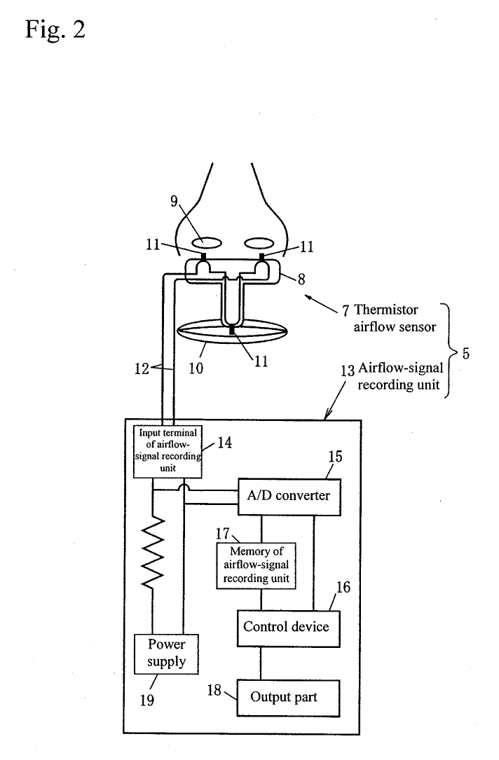

An example of the thermistor respiratory flow meter 5 is shown in FIG. 2. As shown, it comprises a thermistor airflow sensor 7 and an airflow-signal recording unit. The thermistor airflow sensor 7 is positioned near the outlets 9 of the subject's nostrils to detect, by means of a thermistor, any temperature change in the flow of air inhaled and exhaled through breathing. This sensor detects a “flow waveform” (airflow waveform), the details of which are explained later.

FIG. 2 shows a specific structure of the thermistor airflow sensor 7. As shown, it has a T-shaped sensor 8 formed by plastic, etc. This sensor 8 is installed below the subject's nostrils using, for example, double-sided adhesive tape. Thermistor elements 11 are provided in such a way that when the sensor is installed, the elements are positioned close to the three locations, namely, the outlets 9 of right and left nostrils and the oral slit 10 of the subject.

These three thermistor elements 11 are connected in series by lead wires 12. The lead wires 12 can be connected to an input terminal 14 of the airflow-signal recording unit 13, and consequently to a power supply 19 provided inside the airflow-signal recording unit 13.

The thermistor element 11 generates a change in electrical resistance every time it contacts a flow of inhaled or exhaled air (also referred to as “flow” in this Specification), due to the cooling effect of outside air in the case of contact with inhaled air, or due to the warming effect of the subject's body temperature in the case of contact with exhaled air. This change in electrical resistance changes the current (detection current) flowing through the lead wires 12. This current change is input to the airflow-signal recording unit 13 to be detected as voltage change, and the detected voltage change is converted to a digital signal via an A/D converter 15.

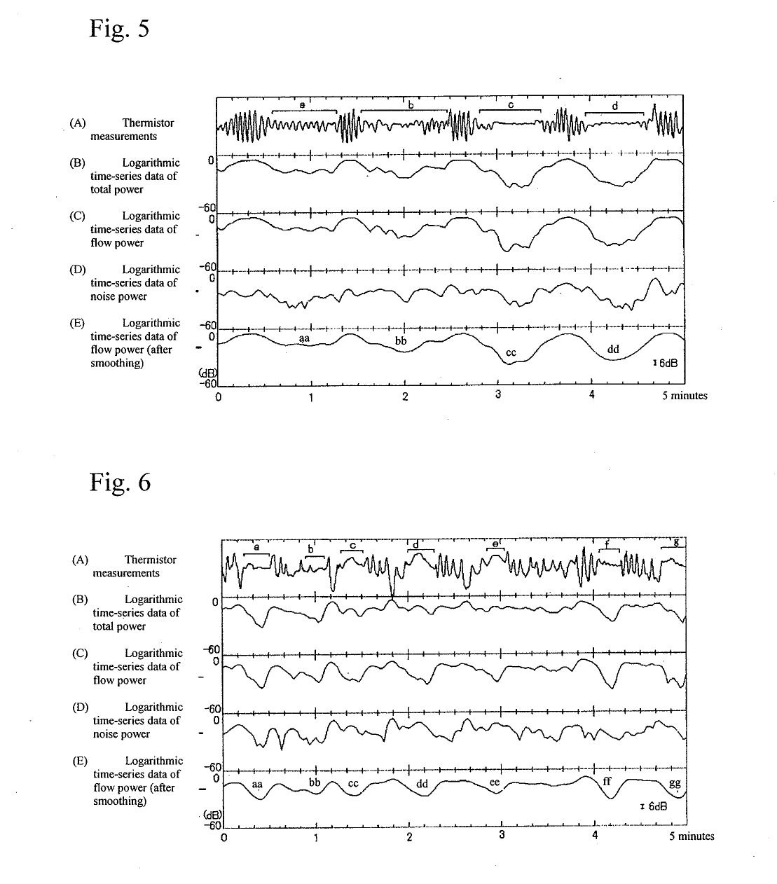

The voltage change detected by the thermistor airflow sensor 7 gives a “flow waveform (airflow waveform),” which is a waveform drawn when change in airflow temperature is plotted as a function of time. FIG. 5 shows data measured over a period of 5 minutes while the subject was sleeping. The “airflow waveform” given by (A) in FIG. 5 is an example of “flow waveform” measured by the thermistor airflow sensor 7. The flow waveform given by (A) in FIG. 5 indicates two occurrences of apnea (indicated by c and d in the figure) and two occurrences of hypopnea (indicated by a and b in the figure) over the 5—minute period.

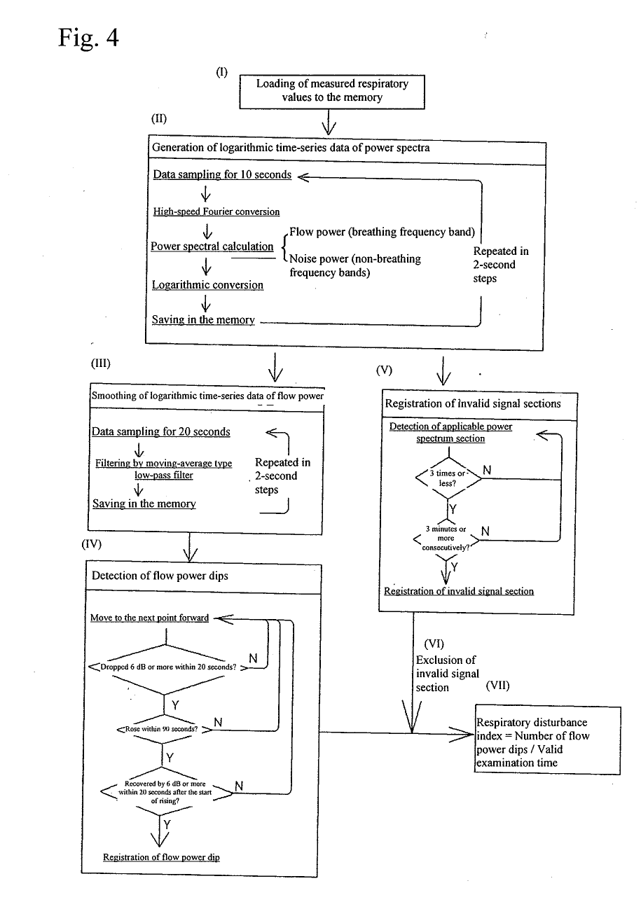

Note that the measured values here actually indicate temperature changes and may not necessarily represent airflow changes caused by breathing, and therefore factors other than breathing may be at play. For this reason, the signal processing shown in FIG. 4 is performed to differentiate factors relating to breathing and factors not relating breathing are differentiated by adding “flow” and “noise,” respectively, as a prefix.

The airflow-signal recording unit 13 comprises an input terminal 14, an A/D converter 15, a control device 16, a memory 17, an output part 18, and a power supply 19. The input terminal 14 is connected to the lead wires 12, through which detection current is input to the airflow-signal recording unit 13. The control device 16 issues commands to the A/D converter 15 to start or end A/D conversion or transfer data to the memory 17.

The A/D converter 15 converts to a digital signal each voltage change detected as a result of change in the aforementioned detection current. For example, voltage changes can be converted to digital airflow waveform data based on a sampling frequency of 10 Hz and 16 quantization bits (this data is hereinafter referred to as “measured values”).

The memory 17 stores the measured values output from the A/D converter 15, while the output part 18 sends the measured values in the memory 17 to the automatic apnea/hypopnea analyzer 2 via, for example, a USB cable. The measured values output from the airflow-signal recording unit 13 may be input to the automatic apnea/hypopnea analyzer 2 via any of the various electronic media available (such as FD, CD or other disc).

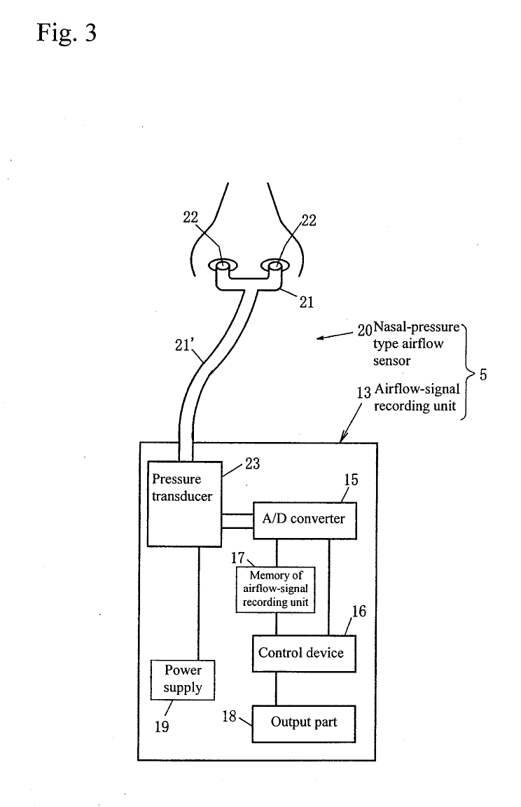

The nasal-pressure type respiratory flow meter 6 comprises a nasal-pressure type airflow sensor 20 and an airflow-signal recording unit 13, as shown inside the imaginary border in FIG. 1. As shown in FIG. 3, the nasal-pressure type airflow sensor 20 comprises a nasal tube 21 having openings 22 corresponding to the positions of the outlets 9 of right and left nostrils. This nasal tube 21 is installed below the nose using double-sided adhesive tape.

These two openings 22 can be connected via a hollow pipe 21′ to a pressure transducer 23 provided inside the airflow-signal recording unit 13. The airflow-signal recording unit 13 has roughly the same structure as the airflow-signal recording unit 13 in the thermistor respiratory flow meter 5.

The flow of air inhaled or exhaled through the openings 22 of the nasal tube 21 is converted to detection current in the piezo-electric transducer 23. The converted detection current is then input to the airflow-signal recording unit 13 to be converted to voltage change, in the same manner as in the thermistor respiratory flow meter 5, after which the voltage change is converted to a digital signal via an A/D converter 15.

The automatic apnea/hypopnea analyzer 2 comprises an I/O interface 24, a bus 25, a CPU 26, and a memory (storage device) 27, as shown in FIG. 1. In actuality, this analyzer is provided as a computer. The I/O interface 24 is used to input measured values from the airflow-signal recording unit 13, and is also connected to the printer 4 to output the measured values and various other data processed by the airflow-signal recording unit 13.

The automatic apnea/hypopnea detection program pertaining to an embodiment of the present invention is stored in the computer's memory 27, to operate the computer as a means for automatically analyzing the measured values for apnea/hypopnea, i.e., as the automatic apnea/hypopnea analyzer 2. The recording medium in which the automatic apnea/hypopnea detection program pertaining to an embodiment of the present invention is stored is a FD, CD or other recording medium containing the program pertaining to an embodiment of the present invention.

Each step, operation and means for automatic analysis of apnea/hypopnea, conducted by the automatic apnea/hypopnea detection program pertaining to an embodiment of the present invention through computer function using measured respiratory flow values, are explained below using FIG. 1 and the flowchart in FIG. 4. The explanation should elucidate the structure and operation of the embodiments of the present invention, especially the structure of the automatic apnea/hypopnea analyzer 2 and the method for automatically detecting apnea/hypopnea. It should also elucidate the details of the automatic apnea/hypopnea detection program and its recording medium pertaining to an embodiment of the present invention, as well as each means or function to be operated by the computer.

1) Loading of Measured Respiratory Flow Values to the Memory (Refer to FIG. 4 (I).)

The automatic apnea/hypopnea analyzer 2 reads measured values from the airflow-signal recording unit 13 via the I/O interface 24 and loads the measured values to the memory 27.

2) Generation of Logarithmic Time-series Data of Flow Power and Logarithmic Time-series Data of Noise Power (Refer to FIG. 4 (II).)

In automatic analysis of apnea/hypopnea, the measured values stored in the memory 27 are processed as follows in the CPU 26.

Measured data over a period of approx. 10 seconds (such as data corresponding to 128 points) are sampled using an appropriate window function (such as the Hanning window function), and high-speed Fourier conversion is performed on the sampled data at a specified interval (such as approx. 2 seconds =20 points) by allowing some overlapping. Through this operation, the intensity of change in measured value, or power (defined as the square mean of signals), is obtained as a power spectrum for each frequency (since the sampling frequency is 10 Hz and the number of data points processed together in high-speed Fourier conversion is 128 in this example, a power spectrum is obtained every 0.078 Hz (10/128)).

Among the obtained power spectra, power spectra belonging to the breathing frequency band (such as 0.133 Hz to 0.5 Hz) are added up (this sum is simply referred to as “flow power” in this Specification) and its time-series data is then obtained, while power spectra belonging to other bands (non-breathing frequency bands) are added up (this sum is simply referred to as “noise power” in this Specification) and its time-series data is then obtained.

Here, the term “breathing frequency band” refers to an appropriate range of frequencies associated with breathing (in consideration of drop in the respiration rate during sleep and quickening of the respiration rate after an apnea, the number of respirations per minute is assumed to change from 8 to 30 as a result of respiratory fluctuation; hence, the breathing frequency band is defined as 0.133 Hz (8 times/min.) to 0.5 Hz (30 times/min.)). “Non-breathing frequency bands” refer to ranges of frequencies that are not considered to have any association with breathing (all frequencies other than the range mentioned above).

To summarize the above, of the intensity of change (power) in measured respiratory flow values the true flow data, or component (flow power) data that is estimated to have resulted from breathing, is obtained separately from other noise component (noise power) data, both as a function of time, to obtain time-series data of flow power and time-series data of noise power.

Then, the values of time-series data of flow power and time-series data of noise power are converted to logarithms to obtain logarithmic time-series data of flow power and logarithmic time-series data of noise power as given by (C) and (D) in FIG. 5. These time-series data are stored in the memory and also shown on the display or any printing medium as time-series curves (refer to (C) and (D) in FIG. 5).

This logarithmic conversion allows the change along the vertical axis to represent not the change in absolute value, but the change in ratio (for example, a drop of 6 dB indicates an amplitude reduction of 50%), and thereby ensuring data accuracy even when the signal levels have changed over a long period of measurement due to positional shifting of the thermistor flow sensor 7, etc. In the logarithmic time-series data of flow power given by (C) in FIG. 5, the flow power drops at points corresponding to occurrences of apnea/hypopnea.

For reference, logarithmic time-series data of the total power before band division (sum of all power spectra in the entire bands) is shown by (B) in FIG. 5. This data was obtained by adding up the power spectra in all frequency ranges obtained by Fourier conversion, without implementing the logarithmic conversion of power spectra. Time-series data of this total power was then obtained and the obtained data was further converted to logarithms. The example given by (B) in FIG. 5 is not much different from the time-series data of flow power covering only the breathing frequency band.

FIG. 6 shows another set of data obtained by the same means explained above and illustrated by (II) of FIG. 4, regarding a set of measured data different from the airflow waveform (flow waveform) given by (A) in FIG. 5. The example given by FIG. 6 shows strong effects of noise components contained in the logarithmic time-series data of total power given by (B) in FIG. 6, and therefore power drops corresponding to occurrences of apnea or hypopnea at c, d and e, among the apnea/hypopnea points of a through g, cannot be grasped. In this example, however, power drops corresponding to all the occurrences of apnea/hypopnea are seen in the logarithmic time-series data of flow power given by (C) in FIG. 6.

As explained above, the effects of noise during measurement can be reduced and thus highly reliable results can be obtained by calculating power spectra (flow power) in the breathing frequency band by means of power spectral analysis. In this stage, the curves of logarithmic time-series data of flow power showing power drops corresponding to occurrences of apnea/hypopnea are not smooth in both the examples of FIGS. 5 and 6, and it is hard to recognize single troughs.

3) Smoothing of Logarithmic Time-series Data of Flow Power (Refer to (III) in FIG. 4.)

Therefore, the logarithmic time-series data of flow power stored in the memory 27 (refer to (C) and (D) in FIG. 5) is processed using a digital filter (for example, data taken over a 20-second period is sampled every 2 seconds and processed using a moving-average type low-pass filter with a cutoff frequency of 0.05 Hz) to obtain smoothed logarithmic time-series data of flow power, which is then stored in the memory 27 (refer to (III) in FIG. 4).

This smoothed logarithmic time-series data of flow power shows each occurrence of apnea or hypopnea as a single, smooth trough, as shown in (E) in FIG. 5 and (E) in FIG. 6.

4) Detection of Flow Power Dips (Refer to (IV) in FIG. 4.)

Next, transitory drops in the airflow power spectrum (referred to as “flow power dips” in this Specification) are obtained under the conditions specified in (IV) of the flowchart given by FIG. 4, from the smoothed logarithmic time-series data of power flow (refer to (E) in FIG. 5).

Specifically, a “flow power dip” is recognized if the power spectrum dropped by a threshold (such as 6 dB) or more within a specified time (such as 20 seconds), started to rise within a specified time (such as 90 seconds) after the start of drop, and recovered by the aforementioned threshold or more within a specified time (such as 20seconds) after the start of rising. A power drop satisfying all these conditions (indicated by “Y” in (IV) of FIG. 4) is registered in the memory (registration of flow power dips in (IV) of FIG. 4). If any of the conditions is not satisfied (indicated by “N” in (IV) of FIG. 4), the applicable power drop is not registered in the memory 27.

This detection operation is implemented by sequentially comparing from the start to end, against the data immediately before and after, each point in all smoothed logarithmic time-series data of flow power stored in the memory in order to detect power drops, and then if any power drop is detected, determining through the program if the subsequent sections satisfy the conditions.

5) Registration of Invalid Signal Sections (Refer to (V) in FIG. 4.)

The automatic apnea/hypopnea analyzer 2 certifies the logarithmic time-series data of flow power obtained by (II) in FIG. 4 (refer to (E) in FIG. 5), based on the distribution and level of power spectra obtained above, in order to determine under the following two conditions if the data is reliable and relating to breathing.

Specifically, the first condition is that the logarithmic time-series data of flow power (logarithmic time-series data covering the total of all power spectra in the breathing frequency band) is equal to or above a specified level (condition 1), while the second condition is that the ratio of flow power and noise power (actually the difference between logarithmic values of flower power and noise power as shown by (C) and (D) in FIG. 5) is equal to or above a specified value (condition 2). Data satisfying both conditions 1 and 2 is certified as reliable data, while data not satisfying either condition is certified as unreliable data.

Even when the data is certified reliable, it may be the case where neither condition 1 nor 2 was satisfied in this certification in an apnea section, but both conditions were satisfied in a normal breathing section before or after the applicable apnea section. Therefore, only when the number of sections that satisfy conditions 1 and 2 for a specified time (such as 3 minutes or more consecutively) is equal to or less than a specified number (such as 3 sections per minute), a judgment of invalid signal section is made and the applicable section is registered as an invalid signal section. Any section that was determined as an invalid signal section is excluded from the evaluation target, even when it contains flow power dip data already registered per FIG. 4 (refer to (VI) in FIG. 4).

For example, flow power and noise power data taken over a 10-second section every 2 seconds are used to certify if the section data to be certified is valid data relating to breathing (for example, data satisfying condition 1 (the flow power is —70 dB or above with the 16-bit value being 0 dB) and condition 2 (the ratio of flow power and noise power is 6 dB or above) is set as valid data).

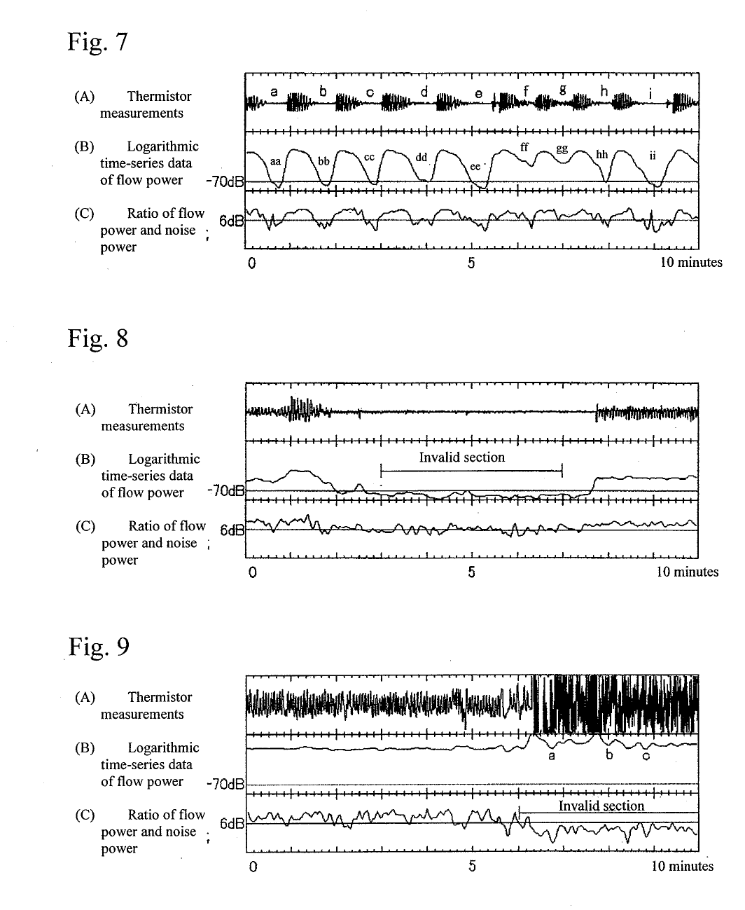

FIG. 7 shows an example where the measured data contains apnea sections. In these apnea sections (a through i), there are areas where neither condition 1 nor 2 is satisfied. However, normal breathing sections in between satisfy both conditions, and at least four sections satisfy both conditions 1 and 2 every minute. Therefore, invalid sections are not recognized, and consequently all nine flow power dips (aa through ii) are adopted.

FIGS. 8 and 9 show examples of bad signals. In FIG. 8, the waveform cannot be recognized as a breathing waveform because the respiratory flow values measured by the thermistor attenuate in the middle. In the attenuated area, the flow power is below —70 dB. Since condition 1 is not satisfied, this data is considered invalid.

FIG. 9 shows an irregular, noise-like waveform where the amplitudes of measured respiratory flow values increase from the middle. This is not considered a breathing waveform. Although there is no drop in flow power in the increased-amplitude area, the ratio of flow power and noise power is less than 6 dB. Since condition 2 is not satisfied, this data is considered invalid. FIG. 9 shows three flow power dips at a, b and c, each in an invalid section. Since all these points occur in an invalid section, they are excluded from the analysis.

6) Generation of Final Analysis Indicator (Refer to (VII) in FIG. 4.)

As the final analysis indicator, the analyzer 2 calculates a respiratory disturbance index using the formula below:

[Respiratory disturbance index] =[Number of flow power dips in valid data sections]/[Time of valid data sections]

The above processing automatically calculates a respiratory disturbance index (number of flow power dips per hour). The automatic apnea/hypopnea analyzer 2 then automatically detects significant apnea/hypopnea if the calculated respiratory disturbance index is equal to or above a specified value. For example, the analyzer does not detect significant apnea/hypopnea if the index is below 5, but it detects significant apnea/hypopnea if the index is 5 or above.

This respiratory disturbance index can also be used to detect the degree of apnea/hypopnea. For example, a respiratory disturbance index of 5 or above but not exceeding 15 indicates a mild case of apnea/hypopnea, while an index of 15 or above but not exceeding 30 indicates a moderate case of apnea/hypopnea. An index of 30 or above suggests a severe case of apnea/hypopnea. The automatic apnea/hypopnea analyzer 2 outputs curves showing measured data and flow power changes to the printer 4.

A respiratory disturbance index is obtained using the automatic apnea/hypopnea detection device, detection method, program and recording medium pertaining to an embodiment of the present invention, and the obtained respiratory disturbance index is used to automatically detect apnea/hypopnea. It is also possible that an embodiment of the present invention is used to perform steps up to calculation of respiratory disturbance index, and a respiratory disturbance index is provided as an estimate value of the apnea/hypopnea index, which is a representative indicator used in sleep polygraph test, in order to confirm that the subject indeed has the sleep apnea syndrome. For example, such an estimate value can indicate a mild case of respiratory disturbance during sleep if the value is 5 or above but not exceeding 15, a moderate case of respiratory disturbance during sleep if the value is 15 or above but not exceeding 30, or a severe case of respiratory disturbance during sleep if the value is 30 or above.

The above explained an embodiment of the automatic apnea/hypopnea detection device, detection method, program and recording medium pertaining to an embodiment of the present invention based on an example. It should be noted, however, that the present invention is not at all limited to this example, and that various examples can be considered within the technical scope specified in the Scope of Claims.

### INDUSTRIAL FIELD OF APPLICATION

The automatic apnea/hypopnea detection device, detection method, program and recording medium pertaining to embodiments of the present invention having the structure explained above, can automatically detect apnea/hypopnea with high reliability via a simple structure and are therefore very useful as a means for providing data with which to check the condition of apnea/hypopnea.

The present application claims priority to Japanese Patent Application No. 2005-296849, filed Oct. 11, 2005, the disclosure of which is herein incorporated by reference in its entirety.

What is claimed is:

1. An automatic apnea/hypopnea detection device comprising a respirometer and an automatic apnea/hypopnea analyzer and automatically detecting apnea/hypopnea based on the airflow waveforms of inhalation and exhalation resulting from breathing of the subject, said automatic apnea/hypopnea detection device characterized by:
the respirometer comprising a flow meter that detects the airflow waveform signals, and an airflow-signal recording unit that converts the airflow waveform signals to digital data as measured values; and
the automatic apnea/hypopnea analyzer comprising:
a means for, by obtaining power spectra from the measured values by Fourier conversion, obtaining time-series data of flow power covering the total of all power spectra belonging to the breathing frequency band among the obtained power spectra as well as time-series data of noise power covering the total of all power spectra belonging to the non-breathing frequency bands among the obtained power spectra, while obtaining logarithmic time-series data of flow power and logarithmic time-series data of noise power from the time-series data of flow power and time-series data of noise power by logarithmic conversion;
a means for smoothing the logarithmic time-series data of flow power;
a means for detecting a flow power dip, which is a transitory drop in the smoothed logarithmic time-series data of flow power;
a means for certifying whether the logarithmic time-series data of flow power before smoothing is valid or invalid under specified conditions;
a means for automatically excluding flow power dips that generated in an invalid section of the logarithmic time-series data of flow power; and
a means for detecting the number of flow power dips occurring per unit valid period of the logarithmic time-series data of flow power, and thereby automatically detecting apnea/hypopnea.

2. The automatic apnea/hypopnea detection device according to claim 1, characterized in that the means for certifying whether the logarithmic time-series data of flow power before smoothing is valid or invalid certifies whether the logarithmic time-series data of flow power before smoothing is valid or invalid under the conditions that the flow power is equal to or above a specified level and the ratio of the flow power and noise power is equal to or above a specified value, and registers as an invalid data section any section of logarithmic time-series data of flow power not satisfying the conditions from among the flow power dips.

3. The automatic apnea/hypopnea detection device according to claim 1, characterized in that the flow meter is a thermistor respiratory flow meter or a nasal-pressure type respiratory flow meter.

4. The automatic apnea/hypopnea detection device according to claim 2, characterized in that the flow meter is a thermistor respiratory flow meter or a nasal-pressure type respiratory flow meter.

5. A method of automatically detecting apnea/hypopnea that detects breathing of the subject using a respirometer and automatically analyzes the digitally converted measured values using an automatic apnea/hypopnea analyzer to automatically detect apnea/hypopnea, said method for automatically detecting apnea/hypopnea characterized by comprising:
a step for, by obtaining power spectra from the measured values by Fourier conversion, obtaining time-series data of flow power covering the total of all power spectra belonging to the breathing frequency band among the obtained power spectra as well as time-series data of noise power covering the total of all power spectra belonging to the non-breathing frequency bands among the obtained power spectra, while obtaining logarithmic time-series data of flow power and logarithmic time-series data of noise power from the time-series data of flow power and time-series data of noise power by logarithmic conversion;
a step for smoothing the logarithmic time-series data of flow power;
a step for detecting a flow power dip, which is a transitory drop in the smoothed logarithmic time-series data of flow power;
a step for certifying whether the logarithmic time-series data of flow power before smoothing is valid or invalid under specified conditions, and registering as an invalid data section any section of logarithmic time-series data of flow power not satisfying the conditions,
a step for automatically excluding, among the flow power dips, those flow power dips that generated in the invalid data sections; and
a step for calculating a respiratory disturbance index as the number of flow power dips occurring per unit time of valid sections excluding the invalid sections.

6. The method of automatically detecting apnea/hypopnea according to claim 5, characterized in that the specified conditions are that the flow power is equal to or above a specified level and that the ratio of the flow power and noise power is equal to or above a specified value.

7. An automatic apnea/hypopnea detection program that is installed in a computer so that it detects breathing of the subject using a respirometer and automatically analyzes the digitally converted measured values in order to automatically detect apnea/hypopnea, said automatic apnea/hypopnea detection program characterized by causing the computer that automatically analyzes apnea/hypopnea to function as:
a means for, by obtaining power spectra from the measured values by Fourier conversion, obtaining time-series data of flow power covering the total of all power spectra belonging to the breathing frequency band among the obtained power spectra as well as time-series data of noise power covering the total of all power spectra belonging to the non-breathing frequency bands among the obtained power spectra, while obtaining logarithmic time-series data of flow power and logarithmic time-series data of noise power from the time-series data of flow power and time-series data of noise power by logarithmic conversion;
a means for smoothing the logarithmic time-series data of flow power;
a means for detecting a flow power dip, which is a transitory drop in the smoothed logarithmic time-series data of flow power;
a means for certifying whether the logarithmic time-series data of flow power before smoothing is valid or invalid under specified conditions, and registering as an invalid data section any section of logarithmic time-series data of flow power not satisfying the conditions;
a means for automatically excluding, among the flow power dips, those flow power dips that generated in the invalid data sections; and
a means for calculating a respiratory disturbance index as the number of flow power dips occurring per unit time of valid sections excluding the invalid sections.

8. The automatic apnea/hypopnea detection program according to claim 7, characterized in that the specified conditions are that the flow power is equal to or above a specified level and that the ratio of the flow power and noise power is equal to or above a specified value.

9. A recording medium that can be read by a computer, in which an automatic apnea/hypopnea detection program according to claim 7 is recorded.

10. A recording medium that can be read by a computer, in which an automatic apnea/hypopnea detection program according to claim 8 is recorded.

11. An automatic apnea/hypopnea detection device comprising:
(a) a respirometer comprising:
a thermistor respiratory flow meter or a nasal-pressure type flow meter configured to sense airflow waveform signals from nostrils; and
an airflow-signal recording unit connected to the thermistor respiratory flow meter or the nasal-pressure type flow meter and configured to convert to digital data the airflow waveform signals and store the converted data as measured respiratory flow values, and
(b) an automatic apnea/hypopnea analyzer configured to obtain power spectra in the breathing frequency band from the measured respiratory flow values, calculate logarithmic time-series data from the power spectra, smooth the data, and then detect transitory drops or flow power dips in the smoothed data, to automatically detect apnea/hypopnea.

12. The automatic apnea/hypopnea detection device according to claim 11, wherein the thermistor respiratory flow meter or the nasal-pressure type flow meter is configured to sense the airflow waveform signals based on airflow waveforms of inhalation and exhalation resulting from breathing of a subject.

13. The automatic apnea/hypopnea detection device according to claim 11, wherein the automatic apnea/hypopnea analyzer is configured to obtain the power spectra from the measured values by Fourier conversion.

14. The automatic apnea/hypopnea detection device according to claim 13, wherein the automatic apnea/hypopnea analyzer is configured to obtain the logarithmic time-series data by (I) obtaining (i) time-series data of flow power covering the total of all power spectra belonging to the breathing frequency band among the obtained power spectra and (ii) time-series data of noise power covering the total of all power spectra belonging to the non-breathing frequency bands among the obtained power spectra, and (II) obtaining logarithmic time-series data of flow power and logarithmic time-series data of noise power from the time-series data of flow power and time-series data of noise power by logarithmic conversion.

15. The automatic apnea/hypopnea detection device according to claim 14, wherein the automatic apnea/hypopnea analyzer is further configured to certify whether the logarithmic time-series data of flow power before smoothing is valid or invalid.

16. The automatic apnea/hypopnea detection device according to claim 14, wherein the automatic apnea/hypopnea analyzer is further configured to automatically exclude the flow power dips generated in an invalid section of the logarithmic time-series data of flow power.

17. The automatic apnea/hypopnea detection device according to claim 14, wherein the automatic apnea/hypopnea analyzer is configured to detect the apnea/hypopnea by detecting the number of flow power dips occurring per unit valid period of the logarithmic time-series data of flow power.

18. The automatic apnea/hypopnea detection device according to claim 15, wherein the automatic apnea/hypopnea analyzer is configured to certify whether the logarithmic time-series data before smoothing is valid or invalid by certifying whether the flow power is equal to or above a specified level and the ratio of the flow power and noise power is equal to or above a specified value.

19. The automatic apnea/hypopnea detection device according to claim 18, wherein the automatic apnea/hypopnea analyzer is further configured to register as an invalid data section any section of logarithmic time-series data of flow power not satisfying the conditions from among the flow power dips.
